(19) United States
(12) Patent Application Publication
(10) Pub. No.: US 2026/0240736 A1
(43) Pub. Date: Aug. 20, 2026

(54) SUNSCREEN COMPOSITIONS FOR PROTECTING SKIN MICROBIOME

(71) Applicant: BASE SE, Ludwigshafen am Rhein (DE)

(72) Inventors: Myriam SOHN, Grenzach-Wyhlen (DE); Manon GAULT, Lyon (FR)

(21) Appl. No.: 19/160,637

(22) PCT Filed: Feb. 28, 2024
(86) PCT No.: PCT/EP2024/055104

§ 371 Date: Aug. 28, 2025

(30) Foreign Application Priority Data
Mar. 1, 2023 (EP) 23305268.7

(51) Int. Cl. A61K 8/41; A61K 8/49; A61Q 17/04

(52) U.S. Cl. CPC A61K 8/411; A61K 8/4966; A61Q 17/04; A61K 2800/48

(57) ABSTRACT

The invention relates to a sunscreen or daily care composition comprising a first UV filter, wherein the first UV filter is hexyl 2-[4-(diethylamino)-2-hydroxybenzoyl]benzoate (INCI diethylamino hydroxybenzoyl hexyl benzoate); and a second UV filter, wherein the second UV filter is selected from the group consisting of (Tris(2-ethylhexyl)-4,4′,4″-(1,3,5-triazin-2,4,6-triyltriimino)tribenzoat) (INCI ethylhexyl triazon), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof for use in a method of protecting the skin microbiome from damage by UV light irradiation.

### TECHNICAL FIELD

The present invention relates to a sunscreen or daily care composition comprising certain UV filters for use in a method of protecting the skin microbiome from damage by UV light irradiation as well as the same sunscreen or daily care composition for use in a method of increasing the viability of the skin microbiome under UV light irradiation and a combination of UV filters for use in a method of protecting the skin microbiome from UV radiation.

### BACKGROUND

UV radiation causes harmful effects on the human skin. Beside the acute effect of sunburn of the skin, UV radiation is also known to increase the risk of skin cancer. Furthermore, long time exposure to UV-A and UV-B light can cause phototoxic and photo allergenic reactions on the skin and can accelerate skin aging.

To protect the human skin from UV radiation, various sun protecting UV filters (also referred to as UV absorbers) exist including UV-A filter (also referred to as UVA filter), UV-B filter (also referred to as UVB filter) and broadband filter. These filters are added to sunscreen or daily care compositions. The UV filters are either organic or inorganic, particulate or non-particulate compounds, of which all have a high absorption efficacy in the UV-light range. In general, UV light relevant to skin health can be divided into UV-A radiation (320-400 nm) and UV-B radiation (290-320 nm). Depending on the position of the absorption maxima, UV filters are divided into UV-A and UV-B filters. In case a UV filter absorbs both, UV-A and UV-B light, it is referred to as a broadband absorber.

Since 2006, the EU commission has recommended that all sunscreens should have a UV-A protection factor which is at least one third of the labelled sun protection factor (SPF), wherein the sun protection factor refers mainly to the UV-B protection. The same should apply for daily care compositions with UV protection to effectively protect the consumer from long term UV damages provided by UV-A radiation.

The human skin comprises millions of bacteria, fungi, and viruses that compose the skin microbiome. These organisms have essential roles in e.g. the protection against invading pathogens. When the barrier, which is provided by these organisms, is broken, humans are vulnerable to invading pathogens. It is known that e.g. compounds comprised in cosmetic products tend to break said barrier.

Furthermore, the skin microbiome is also harmed by UV irradiation directly. UV irradiation impacts the health of the skin microbiome and can greatly reduce the number of the microorganisms that provide the valuable protection of the human skin against pathogens. Therefore, there is a need to find sunscreen compositions that can protect the microorganisms of the skin microbiome effectively from damage by UV light irradiation. At the same time, these sunscreen compositions should be compatible with the skin microbiome.

It was an object of the present invention to provide a sunscreen or daily care composition that protects the skin microbiome from the damage that results from UV light irradiation. It was further an object of the present invention to provide a microbiome friendly sunscreen or daily care composition. Further, it was an object to provide a sunscreen or daily care composition comprising UV filter with a good compatibility with the skin microbiome. Furthermore, it was an object of the invention to provide a sunscreen or daily care composition which increases the viability of the skin microbiome under UV light irradiation.

### SUMMARY OF THE INVENTION

It was surprisingly found that at least one of these objects can be achieved by a sunscreen or daily care composition which comprises
a first UV filter, wherein the first UV filter is hexyl 2-[4-(diethylamino)-2-hydroxybenzoyl]benzoate (INCI diethylamino hydroxybenzoyl hexyl benzoate); and
a second UV filter, wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof.

Surprisingly, a sunscreen or daily care composition comprising this specific combination of UV filters has been found to be well-suited to protect the skin microbiome from UV irradiation. Sunscreen or daily care compositions with different UV filters do not show the same level of protection for the skin microbiome against damages from UV irradiation.

According to a first aspect, the present invention relates to a sunscreen or daily care composition comprising
a first UV filter, wherein the first UV filter is hexyl 2-[4-(diethylamino)-2-hydroxybenzoyl]benzoate (INCI diethylamino hydroxybenzoyl hexyl benzoate); and
a second UV filter, wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
   for use in a method of protecting the skin microbiome from damage by UV light irradiation.

According to a second aspect, the invention relates to a sunscreen or daily care composition comprising
a first UV filter, wherein the first UV filter is hexyl 2-[4-(diethylamino)-2-hydroxybenzoyl]benzoate (INCI diethylamino hydroxybenzoyl hexyl benzoate); and
a second UV filter, wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
   for use in a method of increasing the viability of the skin microbiome under UV light irradiation.

According to a third aspect, the invention relates to a UV filter combination of hexyl 2-[4-(diethylamino)-2-hydroxybenzoyl]benzoate (INCI diethylamino hydroxybenzoyl hexyl benzoate) with 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or (Tris(2-ethylhexyl)-4,4′,4″-(1,3,5-triazin-2,4,6-triyltriimino)tribenzoat) (INCI ethylhexyl triazon) for use in a method of protecting the skin microbiome from UV radiation.

In the following, preferred embodiments of the above aspects are described in further detail. It is to be understood that each preferred embodiment is relevant on its own as well as in combination with other preferred embodiments.

Statements made within this invention with regard to features of the first aspect of the invention also apply to the other aspects of the invention and vice versa. Therefore, specifications of features such as components of the sunscreen or daily care compositions used or the properties of these compositions discussed with regard to one aspect of the invention equally apply to the other aspects of the invention.

### FIGURES

FIG. 1 depicts the results of the viability test for S. epidermidis under different conditions and for different cosmetic formulations.

FIG. 2 depicts the results of the viability test for S. hominis under different conditions and for different cosmetic formulations.

FIG. 3 depicts the results of the viability test for C. acnes under different conditions and for different cosmetic formulations.

### DEFINITIONS

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise” or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “cosmetic composition” refers to any topical product which is intended to be applied to the human body for cleansing, beautifying, promoting attractiveness, altering the appearance, or protecting the skin without affecting the body's structure or function.

Possible cosmetic compositions within the meaning of the invention comprise leave-on and rinse-off products such as face and body care including daily care compositions with or without UV protection and sunscreen or sunscreen compositions, hair care products such as shampoos, conditioners, shower gels, make-up compositions and similar compositions.

The term “sunscreen composition” or “sunscreen” refers to any topical product which absorbs and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

The term “daily care composition” refers to any topical product, which is intended to be used daily without or with a UV protection. In case UV protection is included, it absorbs and may further reflect and scatter certain parts of UV radiation and is used as an everyday care product for the human body, e.g. for face or body. The daily care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care composition are according to the present invention, e.g. leave-on face and body care products.

Suitable leave-on products for face and body are, e.g. sunscreen compositions, decorative preparations, and skin care preparations.

Suitable decorative preparations are, e.g., lipsticks, nail varnishes, eye shadows, mascaras, dry and moist make-up foundations, rouge, powders.

Suitable skin care preparations are e.g., moisturizing, refining, and lifting preparations. The cited leave-on compositions can be in the form of creams, ointments, pastes, foams, gels, lotions, powders, sprays, sticks or aerosols. The daily-care compositions with UV protection protect against damages on human skin by UV light.

The term “UV filter” or “ultraviolet filter” as used herein refers to organic or inorganic compounds, which can absorb and may further reflect and scatter UV radiation caused by sunlight. UV-filter can be classified based on their UV absorbance profile as UV-A, UV-B, or broadband filters. Preferably, the term “UV filter” comprises or consists of any UV filter as defined in the Annex VI (version of 11.11.2022) of the Regulation (EC) No 1223/2009 of the European Parliament and of the Council.

Water soluble UV filters have a solubility in water of at least 2% by weight, preferably at least 3% by weight, more preferably at least 5% by weight.

Oil soluble filters have a solubility in specific emollients of at least 2% by weight, preferably at least 3% by weight, more preferably at least 5% by weight.

The prefix Cn-Cm indicates in each case the possible number of carbon atoms in the group.

The term “C12-C15 alkyl benzoate” refers to esters of benzoic acid with fatty alcohols containing a C12-C15-alkyl chain. C12-C15 alkyl chain is defined as an alkyl chain with C12, C13, C14 or C15 chain length.

The term “Cn-Cm carboxylic acids” as used herein denotes in each case a linear or branched carboxylic acids having from n to m carbon atoms, such as 6 to 24 carbon atoms.

The term “Cn-Cm alcohols” as used herein denotes in each case a linear or branched alcohol having from n to m carbon atoms, such as having from 3 to 24 carbon atoms, or from 6 to 24 carbon atoms, or from 1 to 22 carbon atoms.

The term “C2-C12 dicarboxylic acids” as used herein denotes in each case a dicarboxylic acid having from 2 to 12 carbon atoms such as butanedioic acid (succinic acid), pentanedioic acid (glutaric acid), hexanedioic acid (adipic acid), or decanedioic acid (sebacic acid).

The term “dialkyl ether” as used herein denotes in each case a linear or branched dialkyl ether having a total of from 12 to 36 carbon atoms and comprising at least one ether moiety.

The term “C6-C22 alcohol carbonates” as used herein denotes in each case a linear or branched alcohol carbonates having 6 to 22 carbon atoms and comprising at least one functional group consisting of a carbonyl group flanked by two alkoxy groups.

The term “alkyl” as used herein denotes in each case a straight-chain or branched alkyl group having exemplarily from 1 to 18 carbon atoms. Examples of an alkyl group are methyl, ethyl, n-propyl, iso-propyl, n-butyl, 2-butyl, iso-butyl, tert-butyl, n-pentyl, 1-methylbutyl, 2 methylbutyl, 3 methylbutyl, 2,2-di-methylpropyl, 1 ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethyl-,butyl, 1,2-dimethylbutyl, 1,3-dimethyl-butyl, 2,2-dimethylbutyl, 2,3-dimethylbutyl, 3,3-dimethyl-butyl, 1-ethylbutyl, 2-ethylbutyl, 1,1,2-trimethylpropyl, 1,2,2-trimethylpropyl, 1-ethyl-1-methyl-,propyl, and 1-ethyl-2-methylpropyl.

The term “alkoxy” as used herein denotes in each case a linear or branched alkyl group which is bonded via an oxygen atom and has usually from 1 to 20 carbon atoms. Examples of an alkoxy group are methoxy, ethoxy, n-propoxy, iso-prop-,oxy, n-butyloxy, 2-butyloxy, iso-butyloxy, tert.-butyloxy, and the like.

The term “carboxyalkyl” as used herein includes carboxymethyl, carboxyethyl, carboxypropyl, carboxyisopropyl, carboxybutyl, carboxyisobutyl, carboxyamyl, carboxyhexyl, carboxyheptyl, carboxyoctyl, carboxyisooctyl, carboxynonyl, carboxydecyl, carboxyundecyl, carboxydodecyl, carboxytetradecyl, carboxyhexadecyl, and carboxyoctadecyl, carboxymethyl being preferred.

The term “cycloalkyl” as used herein denotes in each case a monocyclic cycloaliphatic radical having usually from 3 to 10 or from 5 to 8 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “substituted” as used herein means that a hydrogen atom bonded to a designated atom is replaced with a specified substituent, provided that the substitution results in a stable or chemically feasible compound. Unless otherwise indicated, a substituted atom may have one or more substituents and each substituent is independently selected.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. Alternatively, or additionally, the emollients improve the distribution of the product on the skin. Emollients can be divided into different groups depending on their polarity index.

The term “sensitive skin” refers to skin of which the natural barrier function is weakened and has broken due to a trigger. A trigger can be for example cold weather, extremely hot water and critical ingredients, which may be included in sunscreen or daily care compositions.

The term “sun protection factor (SPF)” as used herein indicates how well the skin is protected by a sunscreen composition mainly from UV-B radiation. In particular, the factor indicates how much longer the protected skin may be exposed to the sun without getting a sunburn in comparison to untreated skin. For example, if a sunscreen composition with an SPF of 15 is evenly applied to the skin of a person usually getting a sunburn after 10 minutes in the sun, the sunscreen allows the skilled person to stay in the sun 15 times longer. In other words, SPF 15 means that 1/15 of the burning UV radiation will reach the skin, assuming sunscreen is applied evenly at a thick dosage of 2 milligrams per square centimeter (mg/cm2).

The definition of “broadband” protection (also referred to as broad-spectrum or broad protection) may be based for example on the “critical wavelength” or the UVA-PF. For broadband coverage, UV-B and UV-A protection must be provided. According to the US requirements, a critical wavelength of at least 370 nm is required for achieving broad spectrum protection. Furthermore, it is recommended by the European Commission that all sunscreens or cosmetic compositions should have an UV-A protection factor, which is at least one third of the labelled sun protection factor (SPF), e.g. if the sunscreen composition has an SPF of 30 the UVA protection factor has to be at least 10.

The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e. wavelengths from 290-320 nm, but extends to 370 nm in such a way that 90% of the total area under the protective curve in the UV region are reached at minimum at 370 nm.

The term “administration” refers to the application of a sunscreen or daily care composition to the skin of a person.

### Particular Embodiments of the Invention

The first aspect of the present invention relates to a sunscreen or daily care composition comprising
a first UV filter, wherein the first UV filter is hexyl 2-[4-(diethylamino)-2-hydroxybenzoyl]benzoate (INCI diethylamino hydroxybenzoyl hexyl benzoate); and
a second UV filter, wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
   for use in a method of protecting the skin microbiome from damage by UV light irradiation.

It was surprisingly found that a sunscreen or daily care composition comprising diethylamino hydroxybenzoyl hexyl benzoate (DHHB) in combination with bis-ethylhexyloxyphenol methoxyphenyl triazine (BEMT) and/or ethylhexyl triazone (EHT) protects the skin microbiome effectively from damages by UV light irradiation. Interestingly, this specific combination of UV filters in a sunscreen or daily care composition is better suited for protecting the skin microbiome from damages by UV light irradiation than alternative UV filter combinations.

As above-mentioned the skin microbiome comprises numerous organisms, which are important for protecting e.g. mammal (such as humans) against pathogens. It is known that UV irradiation has a negative effect on said skin microbiome. It is further known that several compounds have a negative effect on said skin microbiome. It is to be understood that “protecting the skin microbiome from damage by UV light irradiation” denotes that the inventive composition comprises a specific UV filter combination which maintains a larger microorganism population when exposed to UV irradiation than is the case for the exact same composition that does not comprise the specific UV filter combination. It is to be understood that “increasing the compatibility of the sunscreen or daily composition with skin microbiome” according to the present invention denotes that the inventive compositions comprising one of the specific UV filters have a larger microorganism population over the exact same composition which merely does not comprise the specific UV filters. Suitable representatives of a microorganism of the skin microbiome are e.g. Cutibacterium acnes, Staphylococcus epidermidis, Staphylococcus hominis, Micrococcus luteus, and Corynebacterium jeikeium.

According to a preferred embodiment, the invention relates to the sunscreen or daily care composition for use in a method of protecting a population of bacteria selected from the group consisting of Cutibacterium acnes, Cutibacterium granulosum, Cutibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium tuberculostearicum, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., and mixtures thereof in the skin microbiome from damage by UV light irradiation.

According to a particularly preferred embodiment, the invention relates to the sunscreen or daily care composition for use in a method of protecting a population of bacteria of Staphylococcus hominis, Staphylococcus epidermidis, Cutibacterium acnes, and mixtures thereof in the skin microbiome from damage by UV light irradiation.

According to another preferred embodiment, the invention relates to the sunscreen or daily care composition for use in a method of protection a population of fungi selected from the group consisting of Malassezia furfur, Malassezia restricta, Malassezia globosa, Candida spp., and mixtures thereof in the skin microbiome from damage by UV light irradiation.

According to another preferred embodiment, the sunscreen or daily care composition further comprises at least one additional UVB filter or broadband filter, preferably selected from the group consisting of 4,4′-[[6-[[4-[[(1,1-dimethylethyl)amino]carbonyl]phenyl]amino]-1,3,5-triazin-2,4-diyl]diimino]bis-benzoesäure-bis(2-ethylhexyl)ester (INCI diethylhexyl butamido triazone or DBT), Phenyl-3H-benzimidazole-5-sulfonic acid (INCI Phenylbenzimidazole Sulfonic Acid or PBSA), dimethicone diethyl benzylmalonate (INCI polysilicone-15), 2-(2H-Benzotriazol-2-yl)-4-methyl-6-[2-methyl-3-[1,3,3,3-tetramethyl-1-[(trimethylsilyl)oxy]-1-disiloxanyl]propyl]phenol (INCI drometrizole trisiloxane) and mixtures thereof. Of these preferred additional UVB and broadband filters, Phenyl-3H-benzimidazole-5-sulfonic acid (INCI Phenylbenzimidazole Sulfonic Acid or PBSA) is particularly preferred.

In another preferred embodiment of the present invention, the sunscreen or daily care composition further comprises at least one organic micronized UV filter selected from the group consisting of 2,2′-methylene bis[6-(2H-benzotriazol-2-yl)-4-(1,1,3,3-tetramethylbutyl)phenol](INCI methylene bis-benzotriazolyl tetramethylbutylphenol), 2,4,6-tris(biphenyl-4-yl)-1,3,5-triazine (INCI Tris-Biphenyl triazine), 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine); preferably 2,2′-Methylenebis[4-(1,1,3,3-Tetramethylbutyl)-6-(2H-Benzotriazol-2-yl)Phenol](INCI methylene bis-benzotriazolyl tetramethylbutylphenol) and/or 2,4,6-tris(biphenyl-4-yl)-1,3,5-triazine (INCI Tris-Biphenyl triazine). In case the sunscreen or daily care composition comprises such an organic micronized UV filter, it is preferable that the at least one organic micronized UV filter is not dispersed in (3R,4S,5S,6R)-2-(Decyloxy)-6-(hydroxymethyl)tetrahydro-2H-Pyran-3,4,5-triol (INCI decyl glucoside).

In a preferred embodiment, the organic micronized UV filter is dispersed in polyglycerol monoalkyl ester. Preferably, the polyglycerol monoalkyl ester is selected from the group consisting of decaglyceryl caprate, decaglyceryl monolaurate, decaglyceryl myristate, decaglyceryl oleate, decaglyceryl stearate, decaglyceryl isostearate, hexaglyceryl cap rate, hexaglyceryl laurate, hexaglyceryl myristate, hexaglyceryl oleate, hexaglyceryl stearate, hexaglyceryl isostearate, pentaglyceryl caprate, pentaglyceryl laurate, pentaglyceryl myristate, pentaglyceryl oleate, pentaglyceryl stearate, pentaglyceryl isostearate, and mixtures thereof, more preferably wherein the at least one polyglycerol monoalkyl ester is decaglyceryl monolaurate (INCI Polyglyceryl-10 Laurate).

According to another preferred embodiment the sunscreen or daily care composition further comprises at least one additional UVA filter, preferably selected from the group consisting of [(3Z)-3-[[4-[(Z)-[7,7-Dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI Terephthalylidene Dicamphor Sulfonic Acid), disodium phenyl dibenzimidazole tetrasulfonate, 1-(4-(1,1-dimethylethyl)phenyl)-3-(4-methoxyphenyl)propane-1,3-dione (INCI Butyl Methoxydibenzoylmethane), and mixtures thereof, in particular 1-(4-(1,1-dimethylethyl)phenyl)-3-(4-methoxyphenyl)propane-1,3-dione (INCI Butyl Methoxydibenzoylmethane).

It is a particularly preferred embodiment the sunscreen or daily care composition is free of zinc oxide (ZnO). Zinc oxide has been found to be harmful to the skin microbiome. In another preferred embodiment, the sunscreen or daily care composition does not contain zinc derivative compounds. According to the present invention, zinc derivative compounds denote every compound that comprises an atom of zinc.

In a preferred embodiment, the sunscreen or daily care composition does not contain a zinc salt. In a particular preferred embodiment, the sunscreen or daily care composition does not contain a zinc salt of pyrrolidone carboxylic acid.

In a preferred embodiment, the sunscreen or daily care composition does not contain parabens.

In a preferred embodiment, the sunscreen or daily care composition does not contain PVP (polyvinylpyrrolidone) polymers and VP (vinylpyrrolidone) derivatives such as vinylpyrrolidone/hexadecene copolymer, VP/Eicosene Copolymer, Triacontanyl PVP.

In a preferred embodiment, the sunscreen or daily care composition does not contain polymers based on acrylic acid such as for example not limited to compounds with the INCI carbomers, C10/30 alkyl acrylate crosspolymer.

According to a preferred embodiment of the invention, the sunscreen or daily care composition comprises hexyl 2-[4-(diethylamino)-2 hydroxybenzoyl]benzoate (INCI diethylamino hydroxybenzoyl hexyl benzoate) in an amount of from 0.1 to 10.0 wt. %, preferably from 0.5 to 8.0 wt. %, and in particular from 1.0 to 5.0 wt. %, based on the total weight of the sunscreen or daily care composition.

According to another preferred embodiment, the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 7.0 wt. %, preferably from 0.5 to 4.0 wt. %, most preferred from 1.0 to 3.0 wt. %, based on the total weight of the sunscreen or daily care composition.

According to another preferred embodiment, the sunscreen or daily care composition comprises (Tris(2-ethylhexyl)-4,4′,4″-(1,3,5-triazin-2,4,6-triyltriimino)tribenzoat) (INCI ethylhexyl triazone) in an amount of from 0.1 to 5.0 wt. %, preferably from 0.5 to 4.0 wt. %, most preferred from 0.8 to 3.0 wt. % based on the total weight of the sunscreen or daily care composition.

In a preferred embodiment, the sunscreen or daily care composition further comprises a dermatologically acceptable emulsifier, thickener, and/or emollient.

Preferred emulsifiers include
glucose derivatives such as cetearyl glucoside, arachidyl glucoside, lauryl glucoside, polyglyceryl-3 methylglucose distearate, methyl glucose sesquistearate;
sucrose derivative such as sucrose polystearate, sucrose palmitate;
sorbitol derivatives;
glycerides of fatty acids such as glyceryl stearate, glyceryl oleate;
glumatic acid derivatives such as sodium stearoyl glutamate;
sulfosuccinic acid derivatives such as disodium cetearyl sulfosuccinate;
phosphoric acid derivatives such as potassium cetyl phosphate;
fatty acid esters of polyglyceryl such as polyglyceryl-3-diisostearate, polyglyceryl-2-dipolyhydroxystearate;

Preferred emollients include
esters of linear or branched fatty acids with linear or branched fatty alcohols such as propylheptyl caprylate, coco caprylate, isopropyl myristate, ethylhexyl palmitate;
esters of aromatic carboxylic acids with linear or branched fatty alcohols such as C12-C15-alkyl benzoate, ethylhexyl benzoate, phenethyl benzoate;
dicarboxylic acid esters with linear or branched alcohols such as dibutyl adipate, dicaprylyl carbonate, diisopropyl sebacate;
esters of hydroxycarboxylic acids with linear or branched fatty alcohols;
esters of linear or branched fatty acids with polyhydric alcohol such as butylene glycol dicaprylate/dicaprate;
mono-, di-, tri-glycerides based on C6-C18 fatty acids such as caprylic/capric triglycerides, coco glycerides;
guerbet alcohols such as octyldodecynol;
hydrocarbons such as hydrogenated polyisobutene, mineral oil, squalene, isohexadecane;
ethers such as dicaprylyl ether;
silicone derivatives (organomodified polysiloxanes) such as dimethylpolysiloxane.

Preferred thickeners include
fatty alcohols such as cetyl alcohol, cetearyl alcohol, behenyl alcohol, stearyl alcohol;
fatty acids such as stearic acid;
fatty acid esters such as myristyl stearate;
C18-38 Alkyl Hydroxystearoyl
waxes such as beeswax, carnauba wax, microcrystalline wax, ceresin, ozocerite, Oriza sativa Rice Bran Wax;
polysaccharides or derivatives such as xanthan gum, guar gum, agar gum, alginates, gellan gum, carraghenan, tara gum;
silicate derivatives such as magnesium silicates;
cellulose derivatives such as hydroxypropyl cellulose.

In one embodiment, the sunscreen or daily care composition further comprises a sensory enhancers, an adjuvants, a preservatives, and/or a perfumes.

Preferred sensory enhancers include
silica;
mica;
starch derivatives such as aluminum starch octenylsuccinate, Tapioca starch, Oryza sativa starch, Zea mays (corn) starch;
dimethicone derivatives;
boron nitride;
Perlite.

Preferred Humectant include
1,2 pentadiol;
1,2 hexandiol;
1,2 octandiol;
1,2 decandiol;
   2-Methyl-1,3-propandiol.

Preferred adjuvants include
tocopherol derivatives;
retinol derivatives;
ascorbic acid derivatives;
bisabolol;
allantoin;
panthenol;
chelating agents (EDTA, EDDS, EGTA, phytic acid, piroctone olamine);
ethylhexyl glycerin;
hydroxyacetophenone;
caprylhydroxymic acid;
propellants such as propane, butane, isobutene, dimethyl ether;
styrene/PVP or styrene acrylamide copolymers;
insect repellants such as butylacetylaminopropionate.

Preferred preservatives include
benzyl alcohol;
zingerone.

Preferred perfumes are selected from the group consisting of limonene, citral, linalool, alpha-isomethylionon, geraniol, citronellol, 2-isobutyl-4-hydroxy-4-methyltetrahydropyrane, 2-tert.-pentylcyclohexylacetate, 3-methyl-5-phenyl-1-pentanol, 7-acetyl-1,1,3,4,4,6-hexamethyltetraline, adipine acid diester, alpha-amylcinnamaldehyde, alpha-methylionon, amyl C butylphenylmehtylpropionalcinnamal, amylsalicylate, amylcinnamylalcohol, anisalcohol, benzoin, benzylalcohol, benzylbenzoate, benzylcinnamate, benzylsalicylate, bergamot oil, bitter
orange oil, butylphenylmethylpropioal, cardamom oil, cedrol, cinnamal, cinnamylalcohol, citronnellylmethylcrotonate, lemon oil, coumarin, diethylsuccinate, ethyllinalool, eugenol, Evernia furfuracea extracte, evernia prunastri extracte, farensol, guajak wood oil, hexylcinnamal, hexylcalicylate, hydroxycitronellal, lavender oil, lemon oil, linaylacetate, mandarine oil, menthyl PCA, methylheptenone, nutmeg oil, rosemary oil, sweet orange oil, terpineol, tonka bean oil, triethylcitrate, vanillin and combinations thereof.

In connection with the above preferred embodiments, it is to be understood that if the sunscreen or daily care composition comprises two or more additives (e.g. emulsifier, emollients, thickeners, sensory enhancers, adjuvants, preservatives, perfumes), combinations of the additives as defined above are also part of the invention.

In connection with the above preferred and particularly preferred embodiments, it is to be understood that the sunscreen or daily care composition may further comprise water.

In case water is present in the sunscreen or daily care composition it is to be understood that it is preferably present in an amount of more than 5% by weight, based on the total weight of the composition. Further, it is to be understood that in case water is present in the sunscreen or daily care composition, the sunscreen or daily care composition can be an oil in water emulsion (O/W emulsion), a water in oil emulsion (W/O emulsion), a gel cream or an oil in gel.

In one embodiment of the present invention, the sunscreen or daily care composition can be provided in different forms, e.g. gels, creams, oils, lotions or in the form of a sprayable product.

The above-outlined sunscreen and daily care compositions are in particular suitable for sensitive skin.

The sunscreen or daily care composition may comprise typical sunscreen or daily care composition ingredients (e.g. emulsifier, thickener, emollient, sensory enhancers, an adjuvants, a preservatives, and/or a perfumes). Preferably, the sunscreen or daily care composition comprises glycerol. Preferably, the sunscreen or daily care composition comprises the glycerol in an amount of from 1 to 10 wt.-%, more preferably from 2 to 8 wt.-% or from 4 to 10 wt.-%, and in particular from 5 to 8 wt.-%, based on the total weight of the sunscreen or daily care composition.

In a preferred embodiment, the sunscreen or daily care has a SPF value of 6 to 50+(according to the EC recommendation), the SPF is determined in silico (eg with the sunscreen simulator, https://sunscreensimulator.basf.com/), or in vivo or in vitro.

According to a preferred embodiment of the invention, the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), 3-Methylbutyl (2E)-3-(4-methoxyphenyl)prop-2-enoate (INCI Isoamyl p-Methoxycinnamate), and/or homosalate. These UV filters have been identified as having a negative impact on human health and/or the environment and are therefore preferably left out of the present compositions.

According to another embodiment, the sunscreen or daily care compositions is for use in a method of protecting the skin microbiome from UV-A radiation and/or UV-B irradiation, preferably from UV-A and UV-B irradiation. The UV filter combination present in the sunscreen or daily care composition for the inventive use is well capable of protecting the skin microbiome from UV-A and/or UV-B radiation.

It is to be understood that the above embodiments of the sunscreen or daily care composition are also applicable for the second aspect and the third aspect of the invention, in particular regarding the at least one additional UVB filter, at least one micronized UV filter, at least one additional UVA filter, further ingredients (e.g. emulsifier, thickener, emollient, sensory enhancers, an adjuvants, a preservatives, and/or a perfumes), and the amounts/weight ratios in the sunscreen or daily care composition.

A second aspect of the present invention relates to a sunscreen or daily care composition comprising
a first UV filter, wherein the first UV filter is hexyl 2-[4-(diethylamino)-2-hydroxybenzoyl]benzoate (INCI diethylamino hydroxybenzoyl hexyl benzoate); and
a second UV filter, wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
   for use in a method of increasing the viability of the skin microbiome under UV light irradiation.

The term “increasing the viability of the skin microbiome under UV light irradiation” herein means that UV light irradiation on a sample of the inventive sunscreen or daily care composition leads to a lower reduction in colonies of skin microbiome microorganisms than is the case for the exact same composition that does not comprise the specific UV filter combination as the inventive sunscreen or daily care compositions. The difference in reduction of colonies of skin microbiome microorganisms is at least 10%, preferably at least 20%, more preferred at least 50% and most preferred at least 90%, in the number of colonies when comparing the two compositions, i.e. a first composition comprising the combination of UV filters according to the invention and a second composition with the same components but without the specific combination of UV filters. The second composition can be free of any UV filter or comprise a different combination of UV filter. The viability of the microorganisms of the skin microbiome is tested by first controlling for the viability of the microorganism in darkness without any UV irradiation and then when applying UV light irradiation, preferably with a UVA irradiation of 10 J/cm² and UVB irradiation of 40 mJ/cm². The number of colonies is determined as CFU/ml (CFU=colony-forming unit). Further details for the method to determine the viability of microorganisms in accordance with the invention are outlined in the examples.

A third aspect of the present invention relates to a UV filter combination of hexyl 2-[4-(diethylamino)-2-hydroxybenzoyl]benzoate (INCI diethylamino hydroxybenzoyl hexyl benzoate) with 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or (Tris(2-ethylhexyl)-4,4′,4″-(1,3,5-triazin-2,4,6-triyltriimino)tribenzoat) (INCI ethylhexyl triazone) for use in a method of protecting the skin microbiome from UV radiation.

Described is also a method of protecting the skin microbiome from damage by UV light irradiation by applying a sunscreen or daily care composition comprising
a first UV filter,
wherein the first UV filter is hexyl 2-[4-(diethylamino)-2-hydroxybenzoyl]benzoate (INCI diethylamino hydroxybenzoyl hexyl benzoate); and
a second UV filter,
wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
   on the skin.

Described is also the use of a sunscreen or daily care composition comprising
a first UV filter,
wherein the first UV filter is hexyl 2-[4-(diethylamino)-2-hydroxybenzoyl]benzoate (INCI diethylamino hydroxybenzoyl hexyl benzoate); and
a second UV filter,
wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
   for protecting the skin microbiome from damage from UV light irradiation.

The method and use also refer to the same sunscreen or daily care composition as used in the aspects of the invention.

### EXAMPLES

The present invention is further illustrated by the following examples.

### Evaluation of SPF

The SPF values were calculated using the BASF sunscreen simulator (https://sunscreensimulator.basf.com/).

Determining the Effect of Sunscreen Composition for Protecting Skin Microbiome Bacteria from UV Irradiation

Bacteria:

Staphylococcus hominis (S. hominis), Staphylococcus epidermidis (S. epidermidis), and Cut/bacterium acnes (C. acnes) were used as bacterial strains of the skin microbiome. Staphylococcus epidermidis strain ATCC 12228 and Staphylococcus hominis ATCC 27844 were obtained from Pasteur Institute in France. These strains were grown in Tryptic Soy Agar and in Tryptic Soy Broth (Biomerieux, France) in aerobic condition. Cutibacterium acnes ATCC 11828 was obtained from Pasteur Institute in France. This strain was grown in Schaedler Agar supplemented by 5% of sheep's blood or in Schaedler Broth supplemented by vitamin K3 at 0.5 g/L (Biomerieux, France) in anaerobic condition.

Test design:

A colony of S. epidermidis or S. hominis were inoculated in Tryptic Soy Broth and incubated at 37° C. for 24 h under continuous stirring at 150 rpm.

After this time, a suspension of 10⁴ UFC/ml of S. epidermidis and 10⁴ UFC/ml of S. hominis have been performed using PBS MgCl2 (lg/L) CaCl2) (1.33 g/L).

For C. acnes, a colony was inoculated in Schaedler Broth supplemented by vitamin K3 (0.5 g/L) at 37° C. for 72 h without stirring. After this time, a suspension of 10⁴ UFC/ml of C. acnes have been performed using PBS MgCa.

100 μl of these bacterial suspensions were spread on a Petri dish containing Tryptic Soy Agar for Staphylococci species and Schaedler Agar for C. acnes. Then, 100 μl of formulation to be tested were spread on the Petri dish previously spread with bacteria. Petri dishes were placed in the dark or in an irradiation chamber for the condition with UV irradiation. An irradiation composed of 10 J/cm² of UVA (320-400 nm) and 40 mJ/cm² of UVB (290-320 nm) was applied to the Petri dishes for the condition with UV irradiation. Then, the Petri dishes were placed in an incubation chamber at a temperature of 37° C., 24 h for S. hominis, 48 h for S. epidermidis in aerobic condition and 96 h for C. acnes in anaerobic condition.

Evaluation:

After this time, colonies were counted, and a CFU/ml was determined for each condition tested. Measurements were repeated 4 times per sample. Statistical analysis was performed using Mann-Whitney Rank Sum test analysis with p<0.05 being determined as a statistically significant difference.

Compositions:

Formulations 1 to XY were manufactured as follows:

The ingredients of part A and part B were provided separated, combined, heated up to 80° C. each while stirring. Subsequently, part A was added to part B while stirring and then the combined mixture was homogenized using ultra turrax type homogenization. The homogenized mixture was left to cool to approximately 30° C. and then the pH value was adjusted to around 6.

The general composition of the formulations tested is shown in table 1

TABLE 1
Formulation vehicle used to test impact of UV irradiation
on microorganism survival; amount in wt. % based
on the total weight of the formulations

| Part | INCI | Amount in wt. % |
|---|---|---|
| A | Glyceryl Stearate SE | 2.00 |
| | Sodium Stearoyl Glutamate | 0.20 |
| | C12-15 Alkyl Benzoate | 10.00 |
| | Dibutyl Adipate | 10.00 |
| | Stearyl Alcohol | 1.50 |
| | Hydrogenated Castor Oil | 2.00 |
| | Phenoxyethanol (and) Ethylhexylglycerin | 1.00 |
| | UV filter combination (see table 2a-c) | 8.00 |
| B | Water | qsp 100% |
| | Glycerin | 8.00 |
| | Xanthan Gum | 0.20 |

For control formulations that are free of UV filters (“Composition w/o UV-Filter”), the amount of the formulation attributed to “UV filter combination” is replaced by water.

Abbreviations are used for the UV filters in the following way:
hexyl 2-[4-(diethylamino)-2-hydroxybenzoyl]benzoate (INCI diethylamino hydroxybenzoyl hexyl benzoate) is “DHHB”
(Tris(2-ethylhexyl)-4,4′,4″-(1,3,5-triazin-2,4,6-triyltriimino)tribenzoat) (INCI ethylhexyl triazone) is “EHT”
2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) is “BEMT”
1-(4-(1,1-dimethylethyl)phenyl)-3-(4-methoxyphenyl)propane-1,3-dione (INCI Butyl Methoxydibenzoylmethane) is “BMDBM”
2-ethylhexyl 2-hydroxybenzoate (INCI ethylhexyl salicylate) is “EHS”

For S. epidermidis, the following UV filter combinations 2a-1 to 2a-4 shown in table 2a were tested:

TABLE 2a
UV-filter combinations used in the formulation shown
in table 1; wt. % is based on the total weight of the
formulations of table 1; inv = inventive UV filter
combination; comp = comparative UV filter combination

| UV-Filter | 2a-1 (inv) | 2a-2 (comp) | 2a-3 (inv) | 2a-4 (comp) |
|---|---|---|---|---|
| (abbreviation) | (wt. %) | (wt. %) | (wt. %) | (wt. %) |
| DHHB | 5.00 | | 5.00 | |
| BMDBM | | 5.00 | | 5.00 |
| EHT | 3.00 | 3.00 | | |
| BEMT | | | 3.00 | 3.00 |

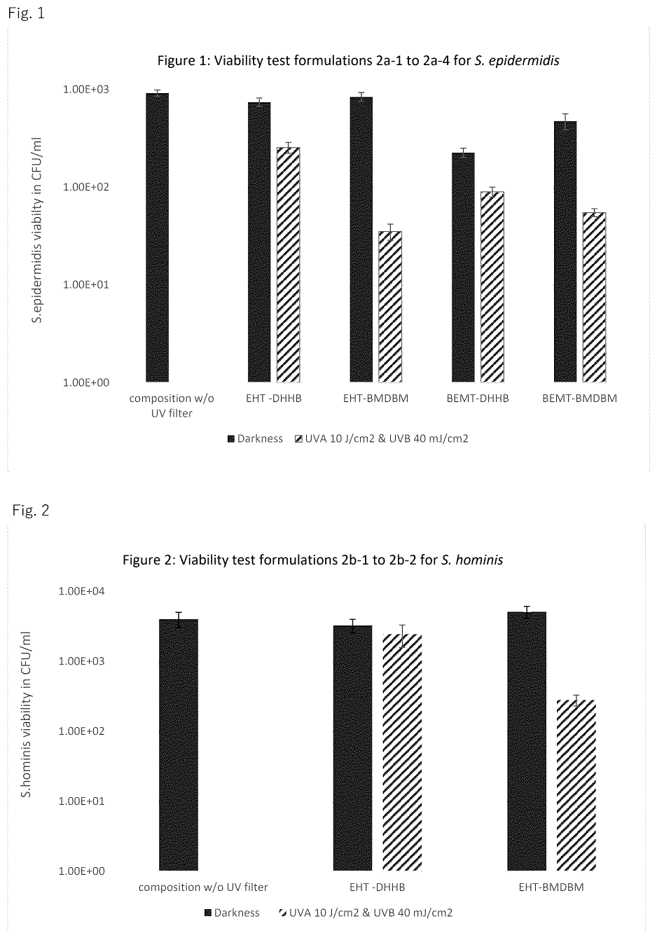

Table 3a and FIG. 1 show the results for S. epidermidis obtained from four measurements (N1-N4) per formulation (Formulations 2a-1 to 2a-4) measured either without any irradiation step (darkness condition) or after UV irradiation (UVA 10 J/cm² and UVB 40 mJ/cm²). The composition without any UV filter protection had a 100% reduction of S. epidermidis colonies under UV irradiation compared to the number of colonies without UV irradiation (in darkness), i.e. all colonies were destroyed by the harmful effect of the irradiation. Both inventive formulations 2a-1 and 2a-3 comprising DHHB and EHT or DHHB and BEMT, respectively, allowed for a statistically significant higher viability of the S. epidermidis under irradiation condition than the two comparative formulations 2a-2 and 2a-4. This effect was particularly pronounced for the combination of EHT and DHHB (formulation 2a-1) as UV filters.

TABLE 3a
Number of S. epidermidis colonies in CFU/ml when combined with different
cosmetic formulations, without and with UV irradiation conditions;
inv = inventive; comp = comparative; SD = standard deviation

| | | | | | Average | |
|---|---|---|---|---|---|---|
| | N1 | N2 | N3 | N4 | N1-N4 | SD |
| In darkness | [CFU/ml] | [CFU/ml] | [CFU/ml] | [CFU/ml] | [CFU/ml] | [CFU/ml] |
| Composition | 9.00 * 10² | 7.30 * 10² | 9.90 * 10² | 1.06 * 10³ | 9.20 * 10² | 7.13 * 10¹ |
| w/o UV-Filter | | | | | | |
| Formulation | 5.70 * 10² | 7.00 * 10² | 9.20 * 10² | 7.80 * 10² | 7.43 * 10² | 7.33 * 10¹ |
| 2a-1 (inv) | | | | | | |
| Formulation | 7.20 * 10² | 6.80 * 10² | 9.00 * 10² | 1.07 * 10³ | 8.43 * 10² | 8.97 * 10¹ |
| 2a-2 (comp) | | | | | | |
| Formulation | 2.73 * 10² | 2.65 * 10² | 1.75 * 10² | 1.90 * 10² | 2.26 * 10² | 2.52 * 10¹ |
| 2a-3 (inv) | | | | | | |
| Formulation | 2.80 * 10² | 3.80 * 10² | 5.60 * 10² | 6.80 * 10² | 4.75 * 10² | 8.96 * 10¹ |
| 2a-4 (comp) | | | | | | |
| Under UV | | | | | | |
| irradiation | | | | | | |
| Composition | 0 | 0 | 0 | 0 | 0 | 0 |
| w/o UV-Filter | | | | | | |
| Formulation | 3.20 * 10² | 1.70 * 10² | 2.90 * 10² | 2.40 * 10² | 2.55 * 10² | 3.28 * 10¹ |
| 2a-1 (inv) | | | | | | |
| Formulation | 1.70 * 10¹ | 4.30 * 10¹ | 3.40 * 10¹ | 4.70 * 10¹ | 3.53 * 10¹ | 6.60 * 10⁰ |
| 2a-2 (comp) | | | | | | |
| Formulation | 1.10 * 10² | 9.50 * 10¹ | 9.30 * 10¹ | 6.00 * 10¹ | 8.95 * 10¹ | 1.05 * 10¹ |
| 2a-3 (inv) | | | | | | |
| Formulation | 4.80 * 10¹ | 7.00 * 10¹ | 4.90 * 10¹ | 5.30 * 10¹ | 5.50 * 10¹ | 5.12 * 10⁰ |
| 2a-4 (comp) | | | | | | |

For S. hominis, the following UV filter combinations 2b-1 to 2b-2 shown in table 2b were tested:

TABLE 2b
UV-filter combinations used in the formulation shown
in table 1; wt. % is based on the total weight of the
formulations of table 1; inv = inventive UV filter
combination; comp: comparative UV filter combination

| UV-Filter | 2b-1 (inv) | 2b-2 (comp) |
|---|---|---|
| (abbreviation) | (wt. %) | (wt. %) |
| DHHB | 5.00 | |
| BMDBM | | 5.00 |
| EHT | 3.00 | 3.00 |

Table 3b and FIG. 2 show the results for S. hominis obtained from four measurements (N1-N4) per formulation (Formulations 2b-1 to 2b-2) measured either without any irradiation step (darkness condition) or after UV irradiation (UVA 10 J/cm² and UVB 40 mJ/cm²). In case of the inventive formulation 2b-1, the number of colonies was almost the same after UV irradiation as it was without UV irradiation which demonstrates how effective the inventive UV filter composition of DHHB and EHT is in protecting the skin microbiome from harmful effects of UV irradiation. In contrast, for the comparative formulation of BMDBM and EHT, a significant drop in viability of the S. hominis colonies was observed in the UV irradiation assays.

TABLE 3b
Number of S. hominis colonies in CFU/ml when combined with different
cosmetic formulations, without and with UV irradiation conditions;
inv = inventive; comp = comparative; SD = standard deviation

| | | | | | Average | |
|---|---|---|---|---|---|---|
| | N1 | N2 | N3 | N4 | N1-N4 | SD |
| In darkness | [CFU/ml] | [CFU/ml] | [CFU/ml] | [CFU/ml] | [CFU/ml] | [CFU/ml] |
| Composition | 6.40 * 10³ | 2.10 * 10³ | 2.80 * 10³ | 4.80 * 10³ | 4.03 * 10³ | 9.77 * 10² |
| w/o UV-Filter | | | | | | |
| Formulation | 4.80 * 10³ | 4.20 * 10³ | 1.80 * 10³ | 2.30 * 10³ | 3.28 * 10³ | 7.25 * 10² |
| 2b-1 (inv) | | | | | | |
| Formulation | 6.10 * 10³ | 6.20 * 10³ | 2.10 * 10³ | 6.10 * 10³ | 5.13 * 10³ | 1.01 * 10³ |
| 2b-2 (comp) | | | | | | |
| Under UV | | | | | | |
| irradiation | | | | | | |
| Composition | 0 | 0 | 0 | 0 | 0 | 0 |
| w/o UV-Filter | | | | | | |
| Formulation | 1.50 * 10³ | 2.10 * 10³ | 1.20 * 10³ | 5.00 * 10³ | 2.45 * 10³ | 8.70 * 10² |
| 2b-1 (inv) | | | | | | |
| Formulation | 4.20 * 10² | 2.80 * 10² | 1.90 * 10² | 2.30 * 10² | 2.80 * 10² | 5.02 * 10¹ |
| 2b-2 (comp) | | | | | | |

Por C. acnes, the following UV filter combinations 2c-1 to 2c-3 shown in table 2c were tested:

TABLE 2c
UV-filter combinations used in the formulation shown
in table 1; wt. % is based on the total weight of the
formulations of table 1; inv = inventive UV filter
combination; comp: comparative UV filter combination

| UV-Filter | 2c-1 (inv) | 2c-2 (comp) | 2c-3 (comp) |
|---|---|---|---|
| (abbreviation) | (wt. %) | (wt. %) | (wt. %) |
| DHHB | 5.00 | | 5.00 |
| BMDBM | | 5.00 | |
| EHT | 3.00 | 3.00 | |
| EHS | | | 3.00 |

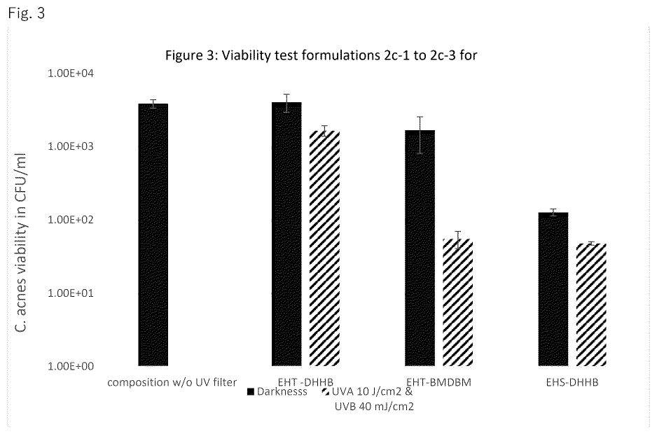

Table 3c and FIG. 3 show the results for C. acnes obtained from four measurements (N1-N4) per formulation (Formulations 2b-1 to 2b-2) measured either without any irradiation step (darkness condition) or after UV irradiation (UVA 10 i/cm² and UVB 40 mi/cm²). Again, the combination of EHT and DHHB proved highly effective at protecting the microbiome bacteria (here: C. acnes) from UV irradiation. In contrast, the comparative formulation 2c-2 comprising EHT and BMDBM as UV filters showed a more than 90% loss in the number of colonies (from more than 10³ in darkness to less than 10² number of colonies under UV irradiation). The viability of C. acnes colonies under UV irradiation was also low in case of the comparative formulation 2c-3 comprising EHS and DHHB. Interestingly, for this UV filter combination, there was also a lack of compatibility with the microorganism before UV irradiation as is apparent from the results in darkness which already showed a significant drop of C. acnes viability compared to the control formulation composition w/o UV filters.

TABLE 3c
Number of C. acnes colonies in CFU/ml when combined with different
cosmetic formulations, without and with UV irradiation conditions;
inv = inventive; comp = comparative; SD = standard deviation

| | | | | | Average | |
|---|---|---|---|---|---|---|
| | N1 | N2 | N3 | N | N1-N4 | SD |
| In darkness | [CFU/ml] | [CFU/ml] | [CFU/ml] | [CFU/ml] | [CFU/ml] | [CFU/ml] |
| Composition | 4.10 * 10³ | 5.20 * 10³ | 3.10 * 10³ | 3.00 * 10³ | 3.85 * 10³ | 5.14 * 10² |
| w/o UV-Filter | | | | | | |
| Formulation | 7.20 * 10³ | 3.80 * 10³ | 3.30 * 10³ | 1.90 * 10³ | 4.05 * 10³ | 1.12 * 10³ |
| 2c-1 (inv) | | | | | | |
| Formulation | 2.40 * 10³ | 3.80 * 10³ | 3.00 * 10² | 2.00 * 10² | 1.68 * 10³ | 8.71 * 10² |
| 2c-2 (comp) | | | | | | |
| Formulation | 1.50 * 10² | 8.80 * 10¹ | 1.43 * 10² | 1.26 * 10² | 1.27 * 10² | 1.39 * 10¹ |
| 2c-3 (comp) | | | | | | |
| Under UV | | | | | | |
| irradiation | | | | | | |
| Composition | 0 | 0 | 0 | 0 | 0 | 0 |
| w/o UV-Filter | | | | | | |
| Formulation | 1.60 * 10³ | 2.40 * 10³ | 1.10 * 10³ | 1.50 * 10³ | 1.65 * 10³ | 2.27 * 10² |
| 2c-1 (inv) | | | | | | |
| Formulation | 6.00 * 10¹ | 9.00 * 10¹ | 5.00 * 10¹ | 2.00 * 10¹ | 5.50 * 10¹ | 1.44 * 10¹ |
| 2c-2 (comp) | | | | | | |
| Formulation | 5.30 * 10^{!} | 4.10 * 10¹ | 4.70 * 10¹ | 5.00 * 10¹ | 4.78 * 10¹ | 2.56 * 10⁰ |
| 2c-3 (comp) | | | | | | |

The results from Examples 2a to 2c demonstrate that the inventive UV filter combination of DHHB and EHT or DHHB and BEMT were highly effective at protecting the skin microbiome from damaging effects of UV irradiation. The differences in Examples 2a to 2c were all statistically significant. The effect on the skin microbiome was herein exemplified by three different types of bacteria (S. epidermidis, S. hominis, C. acnes). Sunscreen or daily care compositions comprising this UV filter combination exhibited a higher level of protection for the skin microbiome from UV irradiation than comparative sunscreen compositions with other UV filter combinations studied. The inventive sunscreen compositions were furthermore compatible with the microorganism of the skin microbiome.

In summary, the inventive compositions are well-suited to protect the skin microbiome from harmful effects of UV irradiation.

1.-14. (canceled)

15. Sunscreen or daily care composition comprising
a first UV filter,
wherein the first UV filter is hexyl 2-[4-(diethylamino)-2-hydroxybenzoyl]benzoate (INCI diethylamino hydroxybenzoyl hexyl benzoate); and
a second UV filter,
wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
for use in a method of protecting the skin microbiome from damage by UV light irradiation.

16. The sunscreen or daily care composition according to claim 15, wherein the sunscreen or daily care composition is for use in a method of protecting a population of bacteria selected from the group consisting of Cutibacterium acnes, Cutibacterium granulosum, Cutibacterium avidum, Staphylococcus epidermidis, Staphylococcus saprophyticus, Staphylococcus hominis, Staphylococcus xylosus, Staphylococcus warneri, Staphylococcus haemolyticus, Staphylococcus saccharolyticus, Staphylococcus conii, Staphylococcus auricularis, Micrococcus luteus, Corynebacterium minutissimum, Corynebacterium jeikeium, Corynebacterium tuberculostearicum, Corynebacterium fastidiosum, Corynebacterium simulans, Corynebacterium amycolatum, Corynebacterium xerosis, Streptococcus mitis, Streptococcus sanguinis, Streptococcus oralis, Brevibacterium spp., Pseudomonas spp., and mixtures thereof in the skin microbiome from damage by UV light irradiation and/or wherein the sunscreen or daily care compositions is for use in a method of protecting a population of fungi selected from the group consisting of Malassezia furfur, Malassezia restricta, Malassezia globosa, Candida spp., and mixtures thereof in the skin microbiome from damage by UV light irradiation.

17. The sunscreen or daily care composition according to claim 15, wherein the sunscreen or daily care composition further comprises at least one additional UVB filter or broadband filter.

18. The sunscreen or daily care composition according to claim 15, wherein the sunscreen or daily care composition further comprises at least one organic micronized UV filter selected from the group consisting of 2,2′-methylene bis[6-(2H-benzotriazol-2-yl)-4-(1,1,3,3-tetramethylbutyl)phenol](INCI methylene bis-benzotriazolyl tetramethylbutylphenol), 2,4,6-tris(biphenyl-4-yl)-1,3,5-triazine (INCI Tris-Biphenyl triazine), 5,6,5′,6′-tetraphenyl-3-3′-(1,4-phenylene)bis(1,2,4-triazine) (INCI phenylene bis-diphenyltriazine), 1,1′-(1,4-piperazinediyl)bis[1-[2-[4-(diethylamino)-2-hydroxybenzoyl]phenyl]-methanone (INCI bis-(diethylaminohydroxybenzoyl benzoyl) piperazine) preferably 2,2′-Methylenebis[4-(1,1,3,3-Tetramethylbutyl)-6-(2H-Benzotriazol-2-yl)Phenol](INCI methylene bis-benzotriazolyl tetramethylbutylphenol) and/or 2,4,6-tris(biphenyl-4-yl)-1,3,5-triazine (INCI Tris-Biphenyl triazine).

19. The sunscreen or daily care composition according to claim 18, wherein the at least one organic micronized UV filter is not dispersed in (3R,4S,5S,6R)-2-(Decyloxy)-6-(hydroxymethyl)tetrahydro-2H-Pyran-3,4,5-triol (INCI decyl glucoside) and/or wherein the at least one organic micronized UV filter is dispersed in polyglycerol monoalkyl ester.

20. The sunscreen or daily care composition according to claim 15, wherein the sunscreen or daily care composition further comprises at least one additional UVA filter.

21. The sunscreen or daily care composition according to claim 15, wherein the sunscreen or daily care composition is free of zinc oxide (ZnO).

22. The sunscreen or daily care composition according to claim 15, wherein the sunscreen or daily care composition comprises hexyl 2-[4-(diethylamino)-2 hydroxybenzoyl]benzoate (INCI diethylamino hydroxybenzoyl hexyl benzoate) in an amount of from 0.1 to 10.0 wt.-%, based on the total weight of the sunscreen or daily care composition and/or wherein the sunscreen or daily care composition comprises 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) in an amount of from 0.1 to 7.0 wt.-% based on the total weight of the sunscreen or daily care composition.

23. The sunscreen or daily care composition according to claim 15, wherein the sunscreen or daily care composition comprises (Tris(2-ethylhexyl)-4,4′,4″-(1,3,5-triazin-2,4,6-triyltriimino)tribenzoat) (INCI ethylhexyl triazone) in an amount of from 0.1 to 5.0 wt.-%, preferably from 0.5 to 4.0 wt.-%, based on the total weight of the sunscreen or daily care composition.

24. The sunscreen or daily care composition according to claim 15, wherein the sunscreen or daily care composition further comprises a dermatologically acceptable emulsifier, thickener, and/or emollient.

25. The sunscreen or daily care composition according to claim 15, wherein the sunscreen or daily care composition does not contain 2-Ethylhexyl 2-cyano-3,3-diphenylprop-2-enoate (INCI Octocrylene), (RS)-2-Ethylhexyl (2E)-3-(4-methoxyphenyl)prop-2-enoate (INCI Ethylhexyl Methoxycinnamate), 3-Methylbutyl (2E)-3-(4-methoxyphenyl)prop-2-enoate (INCI Isoamyl p-Methoxycinnamate), and/or homosalate.

26. The sunscreen or daily care composition according to claim 15, wherein the sunscreen or daily care compositions is for protecting the skin microbiome from UV-A radiation and/or UV-B radiation.

27. Sunscreen or daily care composition comprising
a first UV filter,
wherein the first UV filter is hexyl 2-[4-(diethylamino)-2-hydroxybenzoyl]benzoate (INCI diethylamino hydroxybenzoyl hexyl benzoate); and
a second UV filter,
wherein the second UV filter is selected from the group consisting of (Tris(2-ethylhexyl)-4,4′,4″-(1,3,5-triazin-2,4,6-triyltriimino)tribenzoat) (INCI ethylhexyl triazone), 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine), and mixtures thereof
for use in a method of increasing the viability of the skin microbiome under UV light irradiation.

28. UV filter combination of hexyl 2-[4-(diethylamino)-2-hydroxybenzoyl]benzoate (INCI diethylamino hydroxybenzoyl hexyl benzoate) with 2,4-Bis-{[4-(2-ethyl-hexyloxy)-2-hydroxy]-phenyl}-6-(4-methoxyphenyl)-1,3,5-triazine (INCI bis-ethylhexyloxyphenol methoxyphenyl triazine) and/or (Tris(2-ethylhexyl)-4,4′,4″-(1,3,5-triazin-2,4,6-triyltriimino)tribenzoat) (INCI ethylhexyl triazone) for use in a method of protecting the skin microbiome from UV radiation.
